(19) United States
(12) Patent Application Publication
(10) Pub. No.: US 2003/0208193 A1
(43) Pub. Date: Nov. 6, 2003

(54) Method and system for monitoring fluid temperature during arthroscopic electrosurgery

(76) Inventor: Robert A. Van Wyk, Madeira Beach, FL (US)

Correspondence Address: ROBERT A. VAN WYK, 10801 STARKEY RD. #104-16, LARGO, FL, 33777, US

(21) Appl. No.: 10/138,920

(22) Filed: May 6, 2002

(51) Int. Cl. A61B 18/04

(52) U.S. Cl. 606/34

(57) ABSTRACT

An elongated probe having a temperature sensor at its distal tip is coupled to a monitoring/display unit. Using a cannulated introduction device, the probe is positioned with its distal tip in the intra-articular space such that the temperature sensor is submerged in fluid affected by the electrosurgical procedure. The temperature detected by the sensor is displayed by the monitoring/display unit. A temperature signal from the sensor is compared to a predetermined temperature value, and, if the value is equaled or exceeded, the surgeon is alerted. Similarly, the rate of temperature increase is monitored and compared to a predetermined value of temperature increase, and, if the value is equaled or exceeded, the surgeon is alerted. In another embodiment the temperature sensor is submerged in a fluid stream exiting the joint. In yet another embodiment the monitoring/display unit communicates with an electrosurgical generator such that exceeding a predetermined value for temperature or rate of temperature increase causes the generator to interrupt or reduce its output.

### REFERENCE TO RELATED APPLICATION

This application claims priority to Provisional U.S. Patent application Serial No. 60/287,289 filed on Apr. 30, 2001, entitled “Arthroscopy Fluid Temperature Monitoring System,” hereby incorporated by reference.

### BACKGROUND OF THE INVENTION

The present invention relates to arthroscopic surgery, and more particularly to the monitoring of fluid temperatures during arthroscopic electrosurgery.

Least invasive surgical techniques have gained significant popularity because of their ability to accomplish outcomes with reduced patient pain and accelerated return of the patient to normal activities. Arthroscopic surgery, in which the intra-aticular space is filled with fluid, allows orthopedists to efficiently perform procedures using special purpose instruments designed specifically for arthroscopists. Among these special purpose tools are various manual graspers and biters, powered arthroscopy shaver blades and burs, and electrosurgical devices. During the last several years specialized arthroscopic electrosurgical electrodes called ablators have been developed. Exemplary of these instruments are ArthroWands manufactured by Arthrocare (Sunnyvale, Calif.), VAPR electrodes manufactured by Mitek Products Division of Johnson & Johnson (Westwood, Mass.) and electrodes by Oratec Interventions, Inc. (Menlo Park, Calif.). These ablator electrodes differ from conventional arthroscopic electrosurgical electrodes in that they are designed for the bulk removal of tissue by vaporization rather than the cutting of tissue or coagulation of bleeding vessels. During ablation, electrical current flows from all uninsulated surfaces into the conductive fluid surrounding the electrode. Steam bubbles form at the active electrode and arcing occurs within the bubbles between the electrode and tissue brought into close proximity. All electrodes are capable of ablation, however, the geometries of most standard (non-ablator type) electrodes are not efficient for accomplishing the bulk vaporization of tissue.

During ablation, water within the target tissue is vaporized. Because volumes of tissue are vaporized rather than discretely cut out and removed from the surgical site, the power requirements of ablator electrodes are generally higher than those of other arthroscopic electrosurgical electrodes. The efficiency of the electrode design and the characteristics of the Radio Frequency (RF) power supplied to the electrode affect the power required for ablation. Electrodes with inefficient designs and/or powered by RF energy with poorly suited characteristics will require higher power levels to achieve satisfactory tissue removal rates than those with efficient designs and appropriate generators. Because of these factors the ablation power levels of devices produced by different manufacturers vary widely with some using power levels significantly higher than those commonly used by arthroscopists. Ablator electrode systems from some manufacturers may use up to 280 Watts, significantly higher than the 30 to 70 Watt range generally used by other arthroscopic electrosurgical electrodes.

During arthroscopic electrosurgery all of the radio frequency (RF) energy supplied to the electrode becomes heat thereby raising the temperature of the fluid within the joint and the temperature of tissue in contact with the liquid. Electrodes which operate at high power levels will cause proportionately more heating of the fluid. The temperature of the fluid within the joint is critical since cell death begins to occur at 45 C. The extent of thermal injury is determined by the temperature of the fluid to which the tissue is exposed and the duration of exposure. The relationship between the fluid temperature and the time required to produce thermal injury is highly nonlinear. These injuries occur much more quickly at 55 C than at 45 C, and at 65 C occur in a matter of seconds. Thermal injuries to patients have become much more common with the advent of high powered ablation electrodes.

The temperature rise of fluid within the joint during arthroscopic electrosurgery is determined by the volume of fluid within the joint, the rate of fluid flow through the joint, and the power input to the electrode. This relationship is especially important for the surgeon when performing arthroscopic electrosurgery on small joints such as wrists, ankles or elbows. The volume of these joints is extremely small. Fluid flow through the joint is severely restricted by the small inflow and outflow devices used. This combination of small joint volume and low fluid flow rates can result in rapid fluid temperature rise during electrosurgery. The incidence of patient burns during electrosurgery is much greater for small joint arthroscopy than for other arthroscopic procedures. Use of an ablator-type electrode, with its associated higher power levels compared to conventional electrodes, can cause the surgeon to experience unexpectedly high fluid temperatures and lead to thermal necrosis of tissue within the joint. Reports of second degree burns to the patient's skin caused by fluid draining from the joint are not uncommon.

While the average fluid temperature may be readily estimated if the relevant inputs are known, such information is generally unavailable to the surgeon. And, until the introduction of ablator electrodes, the temperature of the fluid within the joint was not of concern to the surgeon. Fluid temperature is of concern during the use of ablator electrodes due to the higher power levels at which they generally operate and the longer periods of time that they are energized. Standard arthroscopic electrosurgical electrodes are generally energized for only brief periods, generally measured in seconds, while specific tissue is resected or modified, or a bleeder coagulated. In contrast, ablator electrodes are energized for longer periods of time, often as much as several minutes, while volumes of tissue are vaporized.

The temperature distribution within the fluid in the joint is strongly affected by the amount of turbulence created by flow through the joint. With low flow rates there will likely be regions within the joint in which the local fluid temperature is significantly above the average fluid temperature, while at higher flow rates the mixing action created by the flow will cause most regions to be near the average fluid temperature.

Surgeons currently have no method for determining or even estimating the temperature of the fluid in the intra-articular space. The generators of ablations systems marketed by some companies do not directly display the power level in Watts, but rather designate their power levels in arbitrary numbers. To determine actual power levels the surgeon must consult the device users' manual. The volume of the joint and flow rates are also only roughly known. Additionally, surgeons are generally unaware of the link between ablation device power levels, flow rates and intra-articular fluid temperatures.

In order to provide background information so that the invention may be completely understood and appreciated in its proper context, reference is made to a number of prior art devices and patents as follows.

Several electrosurgical systems with temperature monitoring exist. Rita Medical Systems, Inc. (Mountainview, Calif.) markets a system designed for the destruction of non-resectable liver lesions through heating of the tissue by RF energy. An array of sensors monitor temperatures in the tissue at a distance from the active electrode during treatment to ensure that desired temperatures are reached throughout the target area. Similarly, Somnus Medical Technologies, Inc. (Sunnyvale, Calif.) has a system designed for the treatment of obstructive sleep apnea, habitual snoring and chronic nasal obstruction. Treatment consists of shrinking tissue as required for each condition, the shrinkage being accomplished by applying RF energy to the area by means of a needle electrode inserted into the tissue. The needle electrode incorporates a temperature sensing device, feedback from which is used to control the power applied to the electrode. As with the Rita Medical device, the completeness of treatment is ensured by direct measurement of the temperature within the tissue. Both the Rita Medical device and the Somnus device are designed for use in “open” surgery rather than in a fluid-filled joint space. While these devices incorporate temperature monitoring during electrosurgery, they do so at discrete locations within the tissue. This method is not suited to the monitoring of fluid temperatures during arthroscopic electrosurgery.

Two companies market systems which monitor temperatures during arthroscopic electrosurgery. The Vulcan Electrothermal Arthroscopy System by Oratec Interventions, Inc. (Menlo Park, Calif.) and the VAPR II electrosurgical system by Mitek Products Division of Johnson and Johnson (Westwood, Mass.) each have a family of temperature controlled electrodes for the thermal treatment of soft tissue. Rather than tissue vaporization, the probes are designed for the thermal modification of tissue through the application RF energy to the target site. U.S. Pat. No. 5,954,716 to Sharkey, et al. describes the Oratec device. Power supplied to the electrode flows from uninsulated surfaces at the probe distal tip into the surrounding conductive fluid and into any tissue in contact with the uninsulated surfaces. Both the fluid and the tissue are heated by the electrical current flowing through them. Heat from the fluid and tissue heats the probe distal tip in which a temperature sensor is located. The sensor within the probe distal tip provides temperature feedback to the generator for the purpose of maintaining a target temperature at the tip, generally around 65 C. Sufficient power is supplied to the probe to maintain the target temperature at the tip. As with other electrosurgical instruments, all of the power that is supplied to the electrode becomes heat thereby raising the temperature of the fluid within the joint and tissue in contact with the fluid. Because the sensor is located at the probe tip it indicates the temperature in this region only and gives the surgeon no information regarding fluid temperatures in other regions of the joint space. In fact, the temperature feedback is for the purpose of maintaining a temperature at the tip which will quickly cause thermal injury to tissue. No information is supplied to the surgeon regarding the fluid temperature at other locations in the joint. These other temperatures, while less than those at the probe tip, may reach levels sufficient to cause unintended damage to surrounding tissue. The temperature sensor in this device provides no protection from thermal injury to tissue in contact with fluid within the joint but remote from the electrode distal tip. The thermal treatment electrodes and system produced by Mitek Products operate in a similar manner.

U.S. Pat. No. 6,135,999 to Fanton, et al. describes a system consisting of an electrosurgical generator and fluid pump together with a temperature sensor located at the instrument tip to provide temperature monitoring at the tip. The system would also monitor impedance at the electrode tip through current and voltage measurements at the generator. Information from the temperature sensor and impedance calculations would be used to control energy supplied to the probe and flow through the joint so as to maintain desired conditions at the probe tip. The system may increase flow or decrease power in response to an indication by the temperature sensor that the temperature at the treatment site exceeds the desired temperature or alternatively, decrease flow or increase power for an under temperature condition. The system is intended to maintain standard conditions at the probe tip and addresses such problems as the buildup of charred tissue on the electrode. It does not provide a method of monitoring fluid temperature within the joint at locations remote from the treatment site. It provides the surgeon with no information useful for preventing unintended thermal damage to the joint.

Whatever the precise merits, features and advantages of the above cited references, none of them achieves or fulfills the purposes of the fluid temperature monitoring system of the present invention.

Accordingly, it is desirable to provide a system for monitoring intra-articular fluid temperature during arthroscopic electrosurgery. It is further desirable to monitor fluid temperature at sites in the intra-articular space other than in the region of elevated temperature localized at the electrode distal tip. It is further desirable to allow the surgeon to either monitor the fluid at specific locations, or, if this is not practical, to monitor the average fluid temperature within the joint It is further desirable that the temperature be displayed so that the surgeon is able to monitor it visually during surgery. It is further desirable that the monitoring system allows the surgeon to set temperature alarm levels which will trigger visual and audible signals for the surgeon if the fluid temperature reaches or exceeds these values. Additionally, it is desirable that the monitoring system alert the surgeon by a visual or audible signal if the rate of temperature increase exceeds a preset value thereby allowing the surgeon to temporarily halt or modify operation of the device prior to reaching undesirable fluid temperatures And finally, it is desirable for the monitoring system to communicate with an electrosurgical generator so that the generator output is interrupted or reduced if a predetermined temperature or rate of temperature increase is equaled or exceeded.

### SUMMARY OF THE INVENTION

A method and apparatus monitor and display the intra-articular fluid temperature during arthroscopic electrosurgery to prevent inadvertent overheating of the joint. A probe having a temperature sensor located at its distal tip is positioned so that the sensor is submerged in the fluid inside the intra-articular space. A spine needle or other suitable cannulated device is inserted into the joint at a desired location. The temperature sensing probe is inserted into the inner lumen of the device and positioned so that the probe distal tip containing the temperature sensor protrudes beyond the introduction device distal tip in the intra-articular space. The probe supplies a temperature signal to a monitoring/display unit which displays the temperature and compares it to at least one preset value entered by the surgeon. If the temperature sensed by the probe equals or exceeds a preset value, the surgeon is alerted. Similarly, the monitoring/display unit monitors the rate of increase of the temperature sensed by the probe and compares it to a present value. If the rate of temperature increase at the temperature sensing probe equals or exceeds the preset value, the surgeon is alerted.

In another embodiment a temperature sensing probe is placed in the stream of fluid draining from the joint. The monitoring unit functions in the same manner as the previous embodiment.

In yet another embodiment the monitoring unit is incorporated in the electrosurgical generator. In addition to alerting the surgeon when preset temperature or rate of temperature increase values are met or exceeded, the generator output is interrupted or reduced until the sensed temperature or rate of temperature increase falls below the preset values.

### BRIEF DESCRIPTION OF THE DRAWINGS

FIG. 1 is a temperature monitoring system formed in accordance with the principles of this invention.

FIG. 2 is a temperature sensing probe.

FIG. 3 is a side sectional view of the probe distal tip of FIG. 2.

FIG. 4 is a temperature sensing probe assembled to a cannulated introduction device.

FIG. 5 is an expanded view of the distal end of FIG. 4.

FIG. 6 is an alternate embodiment of the temperature monitoring system formed in accordance with the principles of this invention.

FIG. 7 is a temperature sensing probe of FIG. 6.

FIG. 8 is an outflow device with outflow extension and temperature probe attached.

FIG. 9 is a side sectional view of FIG. 8.

FIG. 10 is an alternate embodiment of the temperature monitoring system formed in accordance with the principles of this invention.

### DESCRIPTION OF THE EMBODIMENTS

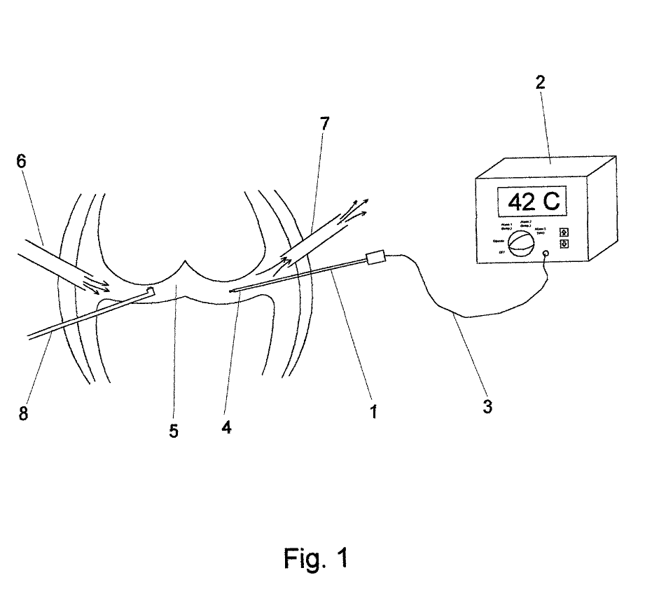

Referring to the drawings, FIG. 1 diagrammatically shows a temperature monitoring system for intra-articular joint fluid temperatures and constructed in accordance with the principles of this invention, consisting of a temperature sensing probe 1 and monitoring/display unit 2 connected by electrical cable 3. Probe 1 is positioned so that its distal end 4 is submerged in intra-articular fluid 5 supplied by inflow device 6 and drained through outflow device 7. An electrosurgical electrode 8 is shown in the intra-articular space.

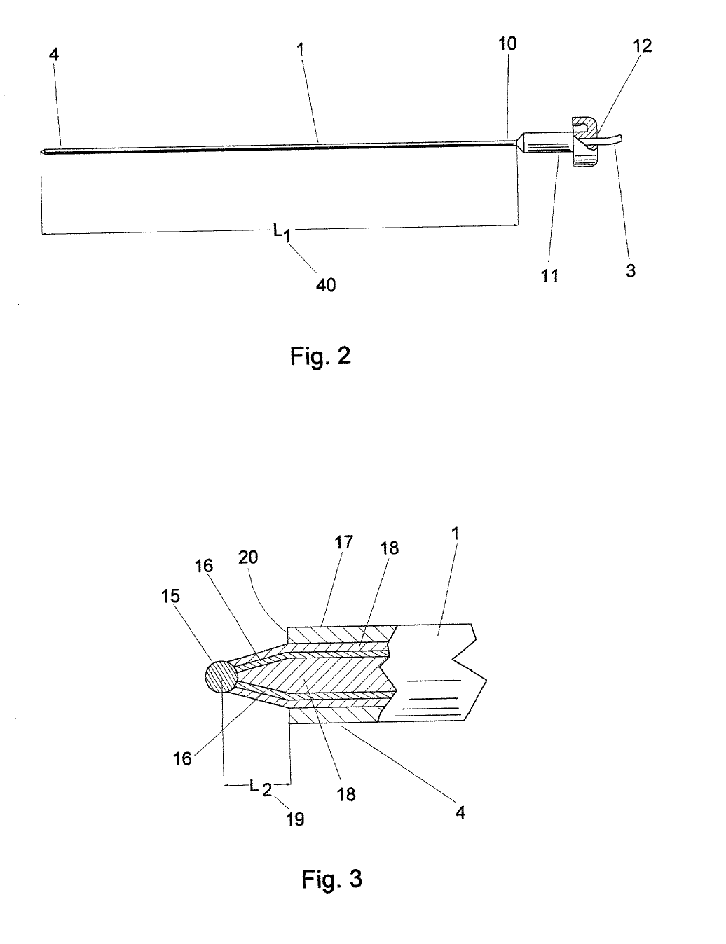

Referring to FIG. 2, temperature sensing probe 1 of length Lsub1 40 has a distal end 4 and a proximal end 10, said proximal end having a hub 11, from the proximal surface 12 of which passes electrical cable 3. Hub 11 has a means for removable mounting of the probe to the proximal end of a suitable cannulated introduction device such as a spine needle.

As is best seen in FIG. 3, distal end 4 of probe 1 has a temperature sensor 15 (thermister, thermocouple, or other) connected by electrical leads 16 which pass through tubular probe body 17 to connect to electrical cable 3 (see FIG. 2). Leads 16 are electrically isolated from tubular probe body 17 by insulating material 18 which rigidly connects sensor 15 to probe body 17. Temperature sensor 15 is displaced distally a distance Lsub2 19 beyond distal surface 20 of probe body 17 and the mass of sensor 15 is minimized so as to minimize the thermal mass and thermal time constant of temperature sensing probe 1.

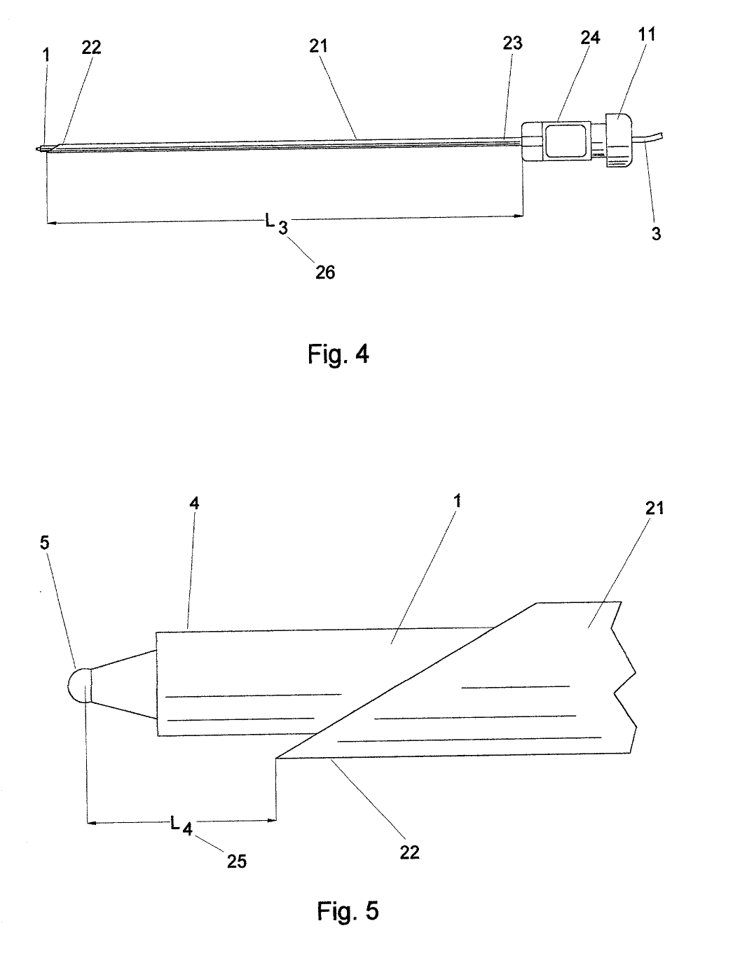

Referring to FIG. 4, cannulated introduction device 21 of length Lsub3 26 having a sharpened distal end 22 and a proximal end 23 has a hub 24 attached to said poximal end having a means for attachment to hub 11 of probe 1. As best seen in FIG. 5, probe 1 is positioned axially within device 21 by probe hub 11 removably mounting to device hub 24 (FIG. 4) such that distal end 4 of probe 1 extends beyond distal end 22 of device 21 a distance Lsub4 25.

Referring again to FIG. 1, monitoring/display unit 2 comprises a means for comparing a measured temperature value at sensor 1 with a predetermined temperature value, and, if the predetermined temperature value is equaled or exceeded, a means for alerting the surgeon. Unit 2 also comprises a means for monitoring the rate of temperature change at the sensor and comparing this rate to a predetermined rate of temperature change, and, if the predetermined rate is equaled or exceeded, a means for alerting the surgeon. Unit 2 also comprises a means for displaying the temperature value at sensor 1 as well as a means for displaying at least one predetermined temperature value and at least one predetermined rate of temperature change. Unit 2 also comprises a means for entering at least one temperature value and at least one value for rate of temperature increase for comparison to values sensor in the manner described previously.

Referring still to FIG. 1, during use the surgeon adjusts fluid flow through inflow device 6 and through outflow device 7 to achieve sufficient fluid pressure within the joint to distend it and create a working space. A suitable location for monitoring the fluid temperature is selected and the cannulated introduction device 21 (FIG. 4) inserted so that its distal tip protrudes into the joint space. Probe 1 is inserted into the lumen of device 21 and hub 11 of probe 1 mounted to hub 22 of device 21 by the aforementioned means. Cable 3 is connected to probe i and monitoring/display unit 2. At least one temperature value and at least one value for rate of temperature change are entered into unit 2. When the surgeon energizes electrode 8 to remove or modify tissue, fluid within the joint is heated with the temperature being determined by certain parameters. These include the volume of the joint, the amount of power supplied to the electrode, the rate of fluid flow through the joint, the temperature of the fluid supplied to the joint, and the length of time that the electrode is energized, the last four parameters being directly controlled by the surgeon. During electrosurgery, the surgeon can observe the fluid temperature on monitoring/display unit 2 to determine the suitability of the controllable parameters selected. To reduce fluid temperature in the joint the surgeon can decrease the power supplied to the electrode, increase the fluid flow rate, supply cooler fluid to the joint, or energize the electrode intermittently for shorter periods of time while allowing the fluid temperature to decrease during periods when the electrode is not energized. If a preset value for temperature or rate of temperature increase is equalled or exceeded, the surgeon is alerted by unit 2 so that he can discontinue energizing of the electrode until the temperature or rate of temperature rise decrease to an acceptable level, that is, to a level which will not cause thermal harm to tissue in contact with fluid in the joint. By so monitoring fluid temperatures, thermal necrosis of tissue within the joint is avoided.

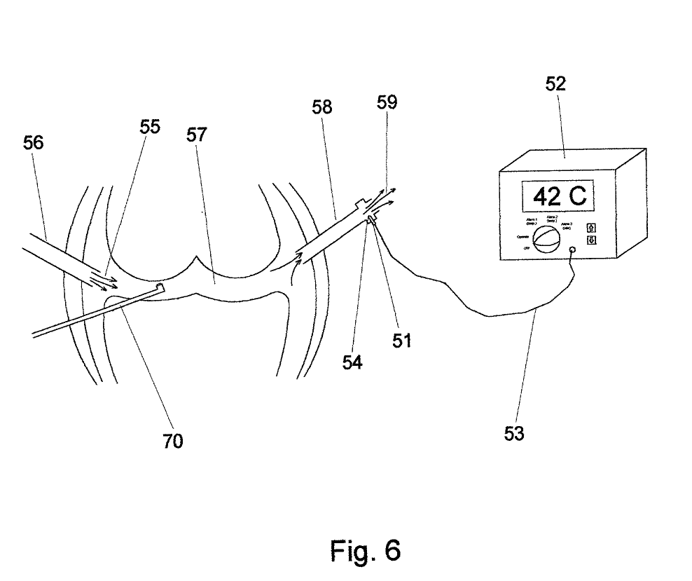

In an alternate embodiment (shown diagramatically in FIG. 6) a temperature sensing probe 61 and monitoring/display unit 52 are connected by an electrical cable 53. Fluid 55 supplied by inflow device 56 fills intra-articular space 57 and is drained through outflow device 58. Probe 51 is positioned so that its distal end 54 is submerged in fluid 59 draining from the intra-articular space. An electrosurgical electrode 70 is shown in the intra-articular space.

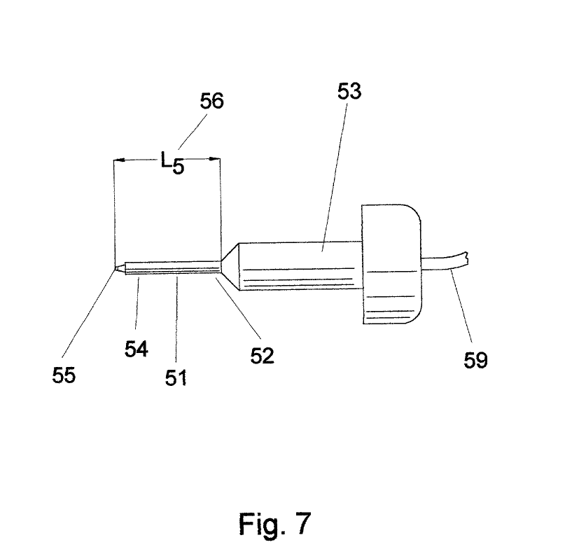

Referring to FIG. 7, probe 51 is constructed in the same manner as probe 1 (FIG. 2) having a proximal end 52 having a hub 53 and a distal end 54 having a temperature sensor 55 at its distalmost tip, except that length Lsub5 56 is less than length Lsub1 40 (FIG. 2).

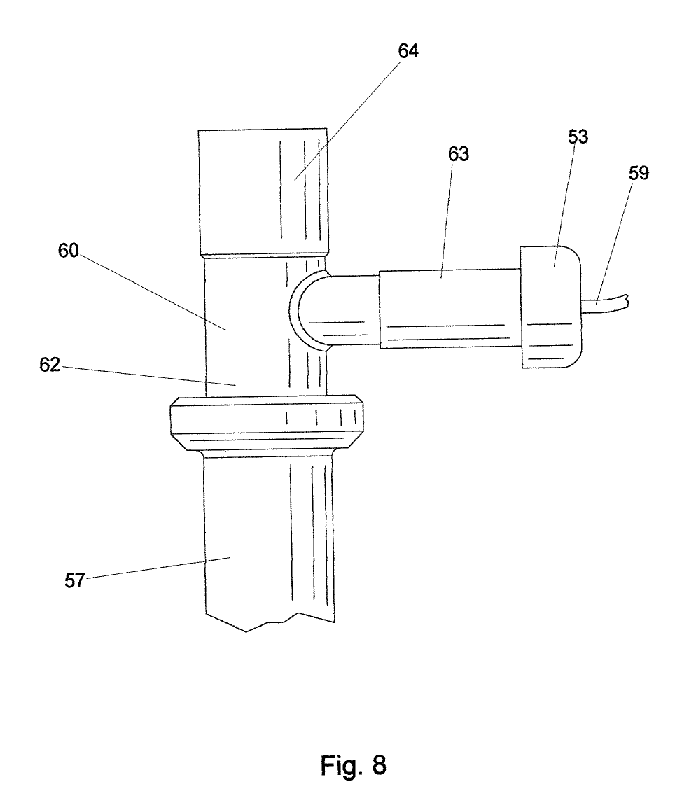

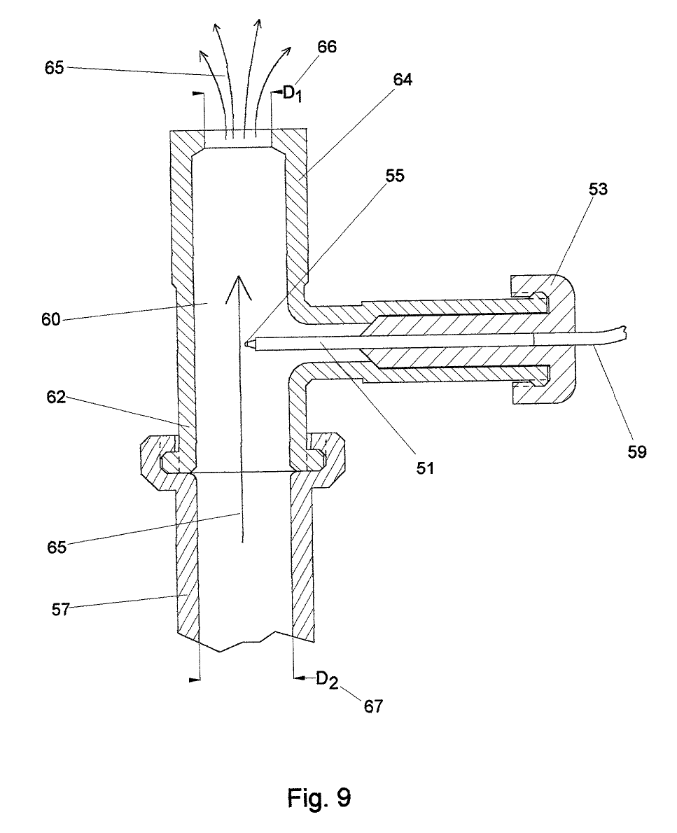

As is best seen in FIGS. 8 and 9, tubular outflow extension 60 has a “T” configuration, one leg 62 of the T having a means for mounting to outflow device 57, a second leg 63 of the “T” having a means for mounting to hub 53 of temperature sensing probe 51 and the third leg 64 of the “T” being open to allow fluid 65 flowing into extension 60 from outflow device 57 to escape. Probe 51 is positioned so that temperature sensor 55 is submerged in fluid 65 flowing from the intra-articular space through outflow device 57. Diameter Dsub1 66 of leg 64 is less than diameter Dsub2 67 of extension 60 to aid in the retention of fluid within extension 60 so as to ensure that sensor 55 is submerged in the fluid.

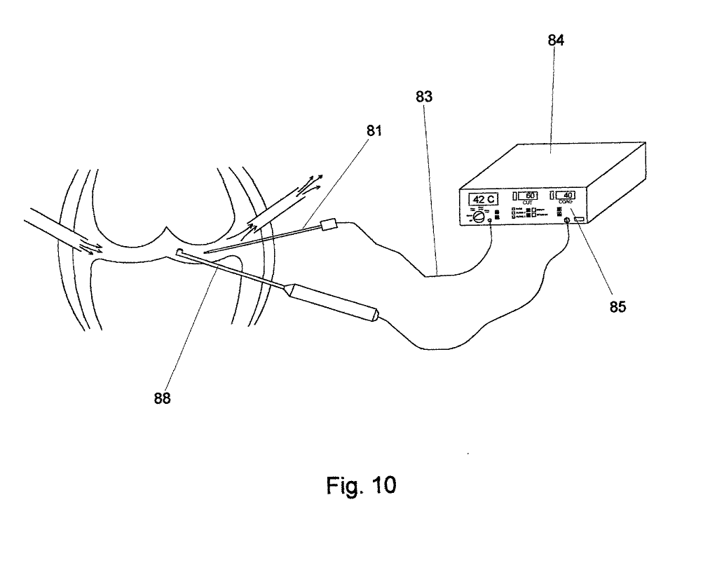

Referring still to FIGS. 9 and 10, during use, outflow extension 60 is mounted to outflow device 57, temperature sensing probe 51 is mounted to outflow extension 57. Referring again to FIG. 6, the surgeon adjusts fluid flow through inflow device 56 to achieve sufficient fluid pressure within the joint to distend it and create a working space. Cable 53 is connected to probe 51 and monitoring/display unit 52. At least one temperature value and at least one value for rate of temperature change are entered into unit 52. When the surgeon energizes electrode 70 to remove or modify tissue, fluid within the joint is heated with the temperature being determined by certain parameters. These include the volume of the joint, the amount of power supplied to the electrode, the rate of fluid flow through the joint, the temperature of the fluid supplied to the joint, and the length of time that the electrode is energized, the last four parameters being directly controlled by the surgeon. During electrosurgery, the surgeon can monitor the fluid temperature to determine the suitability of the controllable parameters selected. To reduce fluid temperature in the joint the surgeon can decrease the power supplied to the electrode, increase the flow rate, supply cooler fluid to the joint, or energize the electrode for shorter periods of time while allowing the fluid temperature to decrease during periods when the electrode is not energized. If a preset value for temperature or rate of temperature increase is equalled or exceeded, the surgeon is alerted by unit 52 so that he can discontinue energizing of the electrode until the temperature or rate of temperature rise decrease to an acceptable level, that is, to a level which will not cause thermal harm to tissue in contact with fluid in the joint. By so monitoring fluid temperatures, thermal necrosis of tissue within the joint is avoided. The surgeon would

In another embodiment, shown diagrammatically in FIG. 10, the circuitry of monitoring/display unit 2 (FIG. 1) is housed in a common enclosure 84 with an electrosurgical generator 85 and communicates with the generator. Construction and operation of temperature sensing probe 81 (FIG. 10) and its associated items are identical to those of probe 1 (FIG. 1). Cable 83 (FIG. 11) is identical in construction and operation to cable 3 (FIG. 1). All aspects and functions of monitoring/display unit 2 (FIG. 1) are present in the monitoring/display circuitry contained in enclosure 84. Additionally, said monitoring/display circuitry has a means for communicating with electrosurgical generator 85 so that if the temperature signal from probe 81 exceeds a preset value, energizing of electrosurgical electrode 88 is interrupted or the level of power supplied to electrode 88 will be reduced until the temperature signal from said probe falls below said preset value. In the same manner, utilizing said means for communicating, if the rate of temperature change detected by said monitoring/display circuitry exceeds a preset value, energizing of electrode 88 is interrupted or the level of power reduced until the detected rate of temperature increase falls below said preset value. Other aspects of generator 85 are unremarkable and well understood by those skilled in the art. Generator 85 may be either monopolar or bipolar.

The foregoing describes a method and system for monitoring intra-articular fluid temperature during arthroscopic electrosurgery at sites in the intra-articular space other than in the region of elevated temperature localized at the electrode distal tip so as to prevent thermal damage to tissue in the joint due to elevated fluid temperatures. The disclosed invention allows the surgeon to either monitor the fluid at specific locations, or, if this is not practical, to monitor the average fluid temperature within the joint by monitoring the outflow fluid temperature. It displays the fluid temperature so that the surgeon can monitor it visually during surgery. It also allows the surgeon to set temperature alarm levels which trigger visual and audible signals if the fluid temperature reaches or exceeds these values. Additionally, it alerts the surgeon by a visual or audible signal if the rate of temperature increase exceeds a preset value thereby allowing the surgeon to temporarily halt or modify operation of the device prior to reaching undesirable fluid temperatures.

While preferred embodiments of the present invention have been shown and described herein, it will be obvious that such embodiments are provided by way of example only. Numerous variations, changes and substitutions will occur to those skilled in the art without departing from the invention herein. Accordingly, it is intended that the invention be limited only by the spirit and scope of the appended claims.

What is claimed is:

1. A method for monitoring intra-articular fluid temperature during arthroscopic electrosurgery, comprising:
providing a probe having a proximal end and a distal end and a temperature sensor located at the distalmost point of said distal end;
positioning said probe so that said distal tip is submerged in the fluid being affected by of the patient's electrosurgical treatment;
providing a monitoring/display unit having a means of displaying a temperature;
providing a means for connecting said probe to said monitoring/display unit such that a temperature signal from said probe is transmitted to said monitoring/display unit; and
displaying the sensed temperature using said means of displaying a temperature.

2. The method of claim 1 wherein said temperature sensor is a thermocouple or thermister.

3. The method of claim 1 wherein said temperature probe comprises a hub attached to said proximal end, said hub comprising a means for attachment to another device.

4. The method of claim 3 wherein said probe is elongated and said distal end protrudes into the intra-articular space of said joint undergoing electrosurgical treatment.

5. The method of claim 4 wherein said probe is introduced into said joint by means of an elongated tubular device having a distal end, a proximal end and an inner lumen of sufficient diameter so that said probe can be inserted into it, said proximal end comprising a hub, and said hub comprising a means for attachment to another device.

6. The method of claim 5 wherein said temperature probe comprises a hub attached to said proximal end;
said hub comprising a means for attachment to another device;
said probe being inserted into the inner lumen of said tubular device; and
said hub of said probe being mounted to said hub of said tubular device so that said probe distal end protrudes beyond said distal end of said tubular device submerged in said fluid.

7. The method of claim 3 wherein said distal end of said probe is submerged in the stream of fluid draining from the joint of the patient undergoing electrosurgery.

8. The method of claim 7 further comprising a tubular outflow device having a distal end and a proximal end;
said distal end being positioned in the intra-articular space so as to allow fluid to flow from said space through said outflow device; and
said proximal end having a first opening to allow fluid to exit said outflow device and a second opening having a means for attachment to another device; and
said temperature probe being positioned within said second proximal opening by said probe hub fastening to said means of attachment of said second opening so that said distal tip of said probe is submerged in fluid flowing through said outflow device.

9. The method of claim 7 further comprising a tubular outflow device having a distal end and a proximal end, said distal end being positioned in the fluid filled intra-articular space so as to allow fluid to flow from said space through said outflow device; and
said proximal end having a means for attachment to another device; and
further comprising a second tubular device having a distal end comprising a means for attachment to another device and a proximal end having a first opening, and a second opening having a means for attachment to another device; and
said distal end of said second tubular device being attached to said proximal end of said outflow device so that a continuous passage is formed to allow drainage of fluid from the intra-articular space, through said outflow device, through said second tubular device and exiting through first proximal end opening of said second tubular device; and
said temperature probe being positioned within said second proximal opening of said second tubular device by said probe hub fastening to said means of attachment of said second opening so that said distal tip of said probe is submerged in fluid flowing through said second tubular device.

10. The method of claim 1 wherein said monitoring/display unit comprises a means for comparing a measured temperature value at said temperature sensor with a predetermined temperature value and further comprises a means for alerting the surgeon if said measured temperature value at the sensor equals or exceeds said predetermined temperature value.

11. The method of claim 10 wherein said monitoring/display unit comprises a means for communicating a signal to another device if said measured temperature value at said sensor equals or exceeds a predetermined temperature value; and
further comprising an electrosurgical generator comprising a means for communication with an external device and a means for interrupting or modifying its output in response to a signal from an external device; and
further comprising a means for transmitting a signal from said monitoring/display unit to said generator so that said generator output is interrupted or reduced if said measured temperature value equals or exceeds a predetermined temperature value, and maintaining said interrupted or reduced power condition until said measured temperature value decreases to below said predetermined value.

12. The method of claim 11 wherein said monitoring/display unit comprising said means associated therewith, and said electrosurgical generator comprising said means associated therewith, are combined into a single unit.

13. The method of claim 1 wherein said monitoring/display unit comprises
a means for determining the rate of increase of measured temperature values at said temperature sensor;
a means for comparing said rate of increase of said measured temperature values at said temperature sensor with a predetermined rate of increase value; and
a means for alerting the surgeon if said determined rate of increase of measured temperature values equals or exceeds said predetermined rate of increase value.

14. The method of claim 13 wherein said monitoring/display unit comprises a means for communicating a signal to another device if said determined rate of increase equals or exceeds said predetermined value; and
further comprising an electrosurgical generator comprising a means for communication with an external device and a means for interrupting or modifiying its output in response to a signal from an external device; and
further comprising a means for transmitting a signal from said monitoring/display unit to said generator so that said generator output is interrupted or reduced if said determined rate of increase equals or exceeds said predetermined value, and maintaining said interrupted or reduced power condition until said determined rate of increase decreases to below said predetermined value.

15. The method of claim 14 in which said monitoring/display unit comprising said means associated therewith, and said electrosurgical generator comprising said means associated therewith, are combined into a single unit.

16. A system for monitoring intra-articular fluid temperature during arthroscopic electrosurgery, comprising:
a probe having a proximal end and a distal end and comprising a temperature sensor located at the distalmost point of said distal end, said probe being positioned so that said sensor is submerged in the fluid being affected by of the patient's electrosurgical treatment;
a monitoring/display unit having a means of displaying a temperature;
a means for connecting said probe to said monitoring/display unit such that a temperature signal from said probe is transmitted to said monitoring/display unit and the temperature displayed by said means of displaying a temperature.

17. The system of claim 16 wherein said temperature sensor is a thermocouple or thermister.

18. The system of claim 16 wherein said temperature probe comprises a hub attached to said proximal end, said hub comprising a means for attachment to another device.

19. The system of claim 18 wherein said probe is elongated and said distal end protrudes into the intra-articular space of said joint undergoing electrosurgical treatment.

20. The system of claim 19 wherein said probe is introduced into said joint by means of an elongated tubular device having a distal end, a proximal end and an inner lumen of sufficient diameter so that said probe can be inserted into it, said proximal end comprising a hub, and said hub comprising a means for attachment to another device.

21. The system of claim 20 wherein said temperature probe comprises a hub attached to said proximal end;
said hub comprising a means for attachment to another device;
said probe being inserted into the inner lumen of said tubular device, and
said hub of said probe being mounted to said hub of said tubular device so that said probe distal end protrudes beyond said distal end of said tubular device submerged in said fluid.

22. The system of claim 18 wherein said distal end of said probe is submerged in the stream of fluid draining from the joint of the patient undergoing electrosurgery.

23. The system of claim 22 further comprising a tubular outflow device having a distal end and a proximal end;
said distal end being positioned in the intra-articular space so as to allow fluid to flow from said space through said outflow device; and
said proximal end having a first opening to allow fluid to exit said outflow device and a second opening having a means for attachment to another device; and
said temperature probe being positioned within said second proximal opening by said probe hub fastening to said means of attachment of said second opening so that said distal tip of said probe is submerged in fluid flowing through said outflow device.

24. The system of claim 22 further comprising a tubular outflow device having a distal end and a proximal end, said distal end being positioned in the fluid filled intra-articular space so as to allow fluid to flow from said space through said outflow device; and
said proximal end having a means for attachment to another device; and
further comprising a second tubular device having a distal end comprising a means for attachment to another device and a proximal end having a first opening, and a second opening having a means for attachment to another device; and
said distal end of said second tubular device being attached to said proximal end of said outflow device so that a continuous passage is formed to allow drainage of fluid from the intra-articular space, through said outflow device, through said second tubular device and exiting through first proximal end opening of said second tubular device; and
said temperature probe being positioned within said second proximal opening of said second tubular device by said probe hub fastening to said means of attachment of said second opening so that said distal tip of said probe is submerged in fluid flowing through said second tubular device.

25. The system of claim 16 wherein said monitoring/display unit comprises a means for comparing a measured temperature value at said temperature sensor with a predetermined temperature value and further comprises a means for alerting the surgeon if said measured temperature value at the sensor equals or exceeds said predetermined temperature value.

26. The system of claim 25 wherein said monitoring/display unit comprises a means for communicating a signal to another device if said measured temperature value at said sensor equals or exceeds a predetermined temperature value; and
further comprising an electrosurgical generator comprising a means for communication with an external device and a means for interrupting or modifying its output in response to a signal from an external device; and
further comprising a means for transmitting a signal from said monitoring/display unit to said generator so that said generator output is interrupted or reduced if said measured temperature value equals or exceeds a predetermined temperature value, and maintaining said interrupted or reduced power condition until said measured temperature value decreases to below said predetermined value.

27. The system of claim 26 wherein said monitoring/display unit comprising said means associated therewith, and said electrosurgical generator comprising said means associated therewith, are combined into a single unit.

28. The system of claim 16 wherein said monitoring/display unit comprises a means for determining the rate of increase of measured temperature values at said temperature sensor, a means for comparing said rate of increase of said measured temperature values at said temperature sensor with a predetermined rate of increase value, and a means for alerting the surgeon if said determined rate of increase of measured temperature values equals or exceeds said predetermined rate of increase value.

29. The system of claim 28 wherein said monitoring/display unit comprises a means for communicating a signal to another device if said determined rate of increase equals or exceeds said predetermined value; and
further comprising an electrosurgical generator comprising a means for communication with an external device and a means for interrupting or modifiying its output in response to a signal from an external device; and
further comprising a means for transmitting a signal from said monitoring/display unit to said generator so that said generator output is interrupted or reduced if said determined rate of increase equals or exceeds said predetermined value, and maintaining said interrupted or reduced power condition until said determined rate of increase decreases to below said predetermined value.

30. The system of claim 29 wherein said monitoring/display unit comprising said means associated therewith, and said electrosurgical generator comprising said means associated therewith, are combined into a single unit.
